(19) United States
(12) Patent Application Publication
(10) Pub. No.: US 2024/0169528 A1
(43) Pub. Date: May 23, 2024

(54) MEDICAL IMAGE PROCESSING DEVICE AND STORAGE MEDIUM STORING MEDICAL IMAGE PROCESSING PROGRAM

(71) Applicant: NIDEK CO., LTD., Gamagori-shi (JP)

(72) Inventors: Ryosuke SHIBA, Gamagori-shi (JP); Tomohiro SASAKI, Gamagori-shi (JP)

(21) Appl. No.: 18/403,357

(22) Filed: Jan. 3, 2024

Related U.S. Application Data
(63) Continuation of application No. PCT/JP2022/023001, filed on Jun. 7, 2022.

(30) Foreign Application Priority Data
Jul. 5, 2021 (JP) 2021-111682

(51) Int. Cl. G06T 7/00; G06N 20/00; G16H 30/20; G16H 30/40

(52) U.S. Cl. CPC G06T 7/0012; G06N 20/00; G16H 30/20; G16H 30/40; G06T 2207/10072; G06T 2207/30041

(57) ABSTRACT

A medical image processing device processes tomographic image data of a living tissue. The medical image processing device includes a control unit that includes at least one processor and at least one memory storing computer program code. The computer program code, when executed by the at least one processor, causes the at least one processor to: acquire a tomographic image in which a layer of the living tissue appears; perform a tilt-reduction process on the acquired tomographic image to reduce a tilt of the layer of the living tissue with respect to a main direction; and acquire medical data by inputting, into a mathematical model, a tilt-reduced image that is the tomographic image on which the tilt-reduction process was performed.

### CROSS REFERENCE TO RELATED APPLICATION

This application is a continuation application of International Patent Application No. PCT/JP2022/023001 filed on Jun. 7, 2022, which designated the U.S. and claims the benefit of priority from Japanese Patent Application No. 2021-111682 filed on Jul. 5, 2021. The entire disclosure of the above application is incorporated herein by reference.

### TECHNICAL FIELD

The present disclosure relates to a medical image processing device that is configured to process tomographic image data of a tissue, and a storage medium storing a medical image processing program executed in the medical image processing device.

### BACKGROUND

There has been known a technology to acquire medical data by inputting medical images into a mathematical model trained by a machine learning algorithm. For example, an ophthalmologic image processing device inputs a base image into a mathematical model to acquire, as medical data, an image with higher quality than the base image. Also, a technology for acquiring, as medical data, analysis results on boundaries of layers of tissue appearing in a medical image.

### SUMMARY

A first aspect of the present disclosure is a medical image processing device that processes tomographic image data of a living tissue. The device includes a control unit that includes at least one processor and at least one memory storing computer program code. The computer program code, when executed by the at least one processor, causes the at least one processor to: acquire a tomographic image in which a layer of the living tissue appears; perform a tilt-reduction process on the acquired tomographic image to reduce a tilt of the layer of the living tissue with respect to a main direction; and acquire medical data by inputting, into a mathematical model, a tilt-reduced image that is the tomographic image on which the tilt-reduction process was performed. The mathematical model is trained by a machine learning algorithm to output medical data by processing an input image.

### BRIEF DESCRIPTION OF DRAWINGS

FIG. 1 is a block diagram showing a schematic configuration of a mathematical model creating device, a medical image processing device, and medical imaging devices.

FIG. 2 is a diagram showing one example of input data and output data when high-quality tomographic image data is output, as medical data, from a mathematical model.

FIG. 3 is a flowchart of a medical image process executed by the medical image processing device.

FIG. 4 is a diagram showing one example of a tomographic image captured by the medical imaging device.

FIG. 5 is a diagram showing a tilt reduced image acquired by performing a tilt-reduction process on the tomographic image shown in FIG. 4.

FIG. 6 is a diagram showing an extracted image in which an image region is extracted from the tilt-reduced image shown in FIG. 5.

FIG. 7 is a diagram showing a high-quality image acquired based on the extracted image shown in FIG. 6.

FIG. 8 is a diagram showing a restored image obtained by performing a restoration process on the high-quality image shown in FIG. 7.

FIG. 9 is a comparative diagram for explaining an advantageous effect according to the present disclosure.

### DESCRIPTION OF EMBODIMENT

Next, a relevant technology will be described first only for understanding the following embodiment.

When a tomographic image of a specific tissue is taken using the same method, layers of the medical image often appear extending along a specific direction (hereinafter, referred to as a “main direction”). However, in a region where the degree of curvature of the layer is large or in a region where a disease exists, the tilt (or an angle) of the direction of the layer with respect to the main direction may become large (in other words, increase). The inventors of the present disclosure have newly discovered that when the tilt of the layer direction with respect to the main direction become large, the accuracy of medical data output by a mathematical model decreased.

One objective of the present disclosure is to provide a medical image processing device and a storage medium storing a medical image processing program to obtain medical data with higher accuracy using a mathematical model trained by a machine learning algorithm.

A first aspect of the present disclosure is a medical image processing device that processes tomographic image data of a living tissue. The device includes a control unit that includes at least one processor and at least one memory storing computer program code. The computer program code, when executed by the at least one processor, causes the at least one processor to: acquire a tomographic image in which a layer of the living tissue appears; perform a tilt-reduction process on the acquired tomographic image to reduce a tilt of the layer of the living tissue with respect to a main direction; and acquire medical data by inputting, into a mathematical model, a tilt-reduced image that is the tomographic image on which the tilt-reduction process was performed. The mathematical model is trained by a machine learning algorithm to output medical data by processing an input image.

A second aspect of the present disclosure is a non-transitory, computer readable storage medium storing a medical image processing program executed by a medical image processing device that processes tomographic image data of a living tissue. The program, when executed by at least one processor of the medical image processing device, causes the at least one processor to: acquire a tomographic image in which a layer of the living tissue appears; perform a tilt-reduction process on the acquired tomographic image to reduce a tilt of the layer of the living tissue with respect to a main direction; and acquire medical data by inputting, into a mathematical model, a tilt-reduced image that is the tomographic image on which the tilt-reduction process was performed. The mathematical model is trained by a machine learning algorithm to output medical data by processing an input image.

A third aspect of the present disclosure is a medical image processing method implemented by a medical image processing device that processes tomographic image data of a living tissue. The method includes: acquiring a tomographic image in which a layer of the living tissue appears; performing a tilt-reduction process on the acquired tomographic image to reduce a tilt of the layer of the living tissue with respect to a main direction; and acquiring medical data by inputting, into a mathematical model, a tilt-reduced image that is the tomographic image on which the tilt-reduction process was performed. The mathematical model is trained by a machine learning algorithm to output medical data by processing an input image.

According to the medical image processing device, the storage medium storing a medical image processing program, and a medical image processing method according to the present disclosure, medical data is acquired with higher accuracy using a mathematical model trained by a machine learning algorithm.

The medical image processing device exemplified in the present disclosure processes data of tomographic images of living tissues. The control unit of the medical image processing device executes an image acquisition step, a tilt-reduction step, and a medical data acquisition step. At the image acquisition step, the control unit acquires a tomographic image in which the tissue layers appear. At the tilt-reduction step, the control unit performs a tilt-reduction process on the tomographic image to reduce the tilt of the layer with respect to the main direction. At the medical data acquisition step, the control unit acquires medical data by inputting a tilt reduced image that is made via the tilt-reduction process at the tilt-reduction step into a mathematical model. The mathematical mode has been trained by a machine learning algorithm and is configured to output medical data by processing input images.

According to the present disclosure, a tilt-reduced image in which the tilt of the layer with respect to the main direction is reduced is input into a mathematical model. As a result, compared to a situation where a tomographic image is directly input into a mathematical model without performing the tilt-reduction process on the tomographic image, it is possible to avoid decreasing the accuracy of the medical data due to the tilt of the layer with respect to the main direction. Therefore, the medical data can be acquired with higher accuracy.

As described above, if a tomographic image of a specific tissue is taken using the same method, the layers of the medical image often appear extending along a specific direction (i.e., the main direction). Therefore, most layers or most parts of the layers of the plurality of medical images used for training the mathematical model have a small tilt (or a small angle) with respect to the main direction. Therefore, for a mathematical model trained with multiple medical images, processing can be performed with high accuracy on layers with small tilts with respect to the main direction, while processing tends to be performed with lower accuracy for layers with large tilts with respect to the main direction. For example, it is possible to improve the accuracy of processing for layers with large tilts by adjusting the network structure (e.g., a filter structure, etc.) of the mathematical model. However, in this case, since the number of parameters increases due to adjustment of the network structure, the number of medical images required for learning, processing time, etc. would also increase. Alternatively, processing accuracy may be improved by including a large number of medical images having layers with large tilts with respect to the main direction in the medical images used for training the mathematical model. However, it would be very time-consuming to prepare a large number of medical images of layers with large tilts with respect to the main direction. In contrast, according to the present disclosure, medical data is acquired with high accuracy through simple processing without re-developing a mathematical model.

The main direction is a direction in which layers of tissue that appear in a tomographic image generally extends when a tomographic image of a specific tissue is taken. In this disclosure, a case will be exemplified in which medical data is acquired from a tomographic image of fundus tissue. In this case, the fundus tissue layer shown in the tomographic image usually extends in a direction (referred to as an X direction in this disclosure) perpendicular to the depth direction of the tissue (referred to as a Z direction in this disclosure). Therefore, the main direction in this disclosure is the X direction. However, the main direction may be set as appropriate depending on the tissue whose tomographic image is to be imaged, the imaging method, and the like. Therefore, the main direction is not necessarily limited to the X direction. For example, the main direction in a three-dimensional tomographic image may be a direction (an X-Y direction) perpendicular to the depth direction (i.e., the Z direction) of the tissue.

Various devices may be used as the imaging device that captures (generates) tomographic images. For example, an OCT device that captures tomographic images of tissues using the principle of optical coherence tomography may be used. In this case, the tomographic image may be, for example, a motion contrast image (for example, an OCT angiography image) acquired from a plurality of OCT signals acquired at different times from the same position in the retinal layer of the fundus. Furthermore, an MRI (magnetic resonance imaging) device, a CT (computed tomography) device, or the like may be used. The tomographic image acquired at the image acquisition step may be a two-dimensional tomographic image or a three-dimensional tomographic image.

The mathematical model may be trained with training data that includes reduced-tilt images in which the tilts of the layers with respect to the main direction are reduced. In this case, the mathematical model may perform processing with higher accuracy on the tomographic image in which the layer tilt has been reduced during the tilt-reduction step.

When an image is input, the mathematical model may output, as medical data, high-quality image data with improved quality with respect to the input image. In this case, even if the image is of a layer having a large angle with respect to the main direction, the image quality is improved to the same level as the image of a layer having a small angle (or tilt) with respect to the main direction.

The mathematical model is not necessarily limited to a mathematical model that outputs high-quality image data as medical data. For example, the mathematical model may perform an analysis process on at least one of a specific structure and a specific disease shown in the tomographic image, and output data indicative of analysis results as medical data. When the tomographic image is an ophthalmological image of a subject's eye, analysis results of at least one of layers of the fundus tissue of the subject's eye, boundaries of the layers of the fundus tissue, an optic disc present in the fundus, layers of an anterior segment tissue, boundaries of the layers of the anterior segment tissue, and a diseased area of the subject's eye may be output. Further, the mathematical model may perform an automatic diagnosis process on the tissue shown in the tomographic image, and output data indicative of automatic diagnosis results as medical data. Further, the mathematical model may output reliability information indicative of reliability of processing (for example, structure or disease analysis processing, etc.) performed on the input medical image as medical data. “Reliability” may be degree of certainty of tomographic image processing using a mathematical model or may be the reciprocal of the low degree of certainty (may also be expressed as “uncertainty”).

The control unit may further perform a restoration step. At the restoration step, the control unit performs a process on the medical data acquired at the medical data acquisition step that is an opposite process to the process that was performed at the tilt-reduction step so that the arrangement of the medical data is restored to the arrangement prior to the tilt-reduction step. In this case, the arrangement of the medical data is appropriately restored to the arrangement of the tissue that was actually captured. Therefore, is reduced, and medical data with a reduced influence due to the tilt of the layers and with appropriate arrangement can be obtained.

Note that when the medical data is data such as a high-quality image, the arrangement of the medical data to be restored may be the arrangement of tissues shown in the image. If the medical data is analysis results of a structure (for example, the analysis results of layer boundaries, etc.), the arrangement of the medical data to be restored may be the arrangement of the analyzed structure.

However, if the arrangement of the medical data is not important (for example, if it is sufficient to only acquire a value such as a reliability level as the medical data), the restoration step may be omitted.

The control unit may further execute an image region extraction step of extracting, from the tomographic image, an image region in which tissue appears. The control unit may input, into the mathematical model, the tomographic image in which the tilt of the layer has been reduced and the image region has been extracted. In this case, compared to a situation where the tomographic image is input into the mathematical model without extracting the image region, the amount of calculations for processing by the mathematical model can be appropriately reduced.

Note that when the control unit extracts the image region and then acquires the medical data, the control unit may perform a process of restoring regions other than the extracted image region on the acquired medical data. In this case, the size of the acquired medical data is appropriately returned to the size of the tomographic image before the image region was extracted. It is also possible to input the tomographic image into the mathematical model without performing the image region extraction step.

At the tilt-reduction step, the control unit may reduce the tilt of the layer by moving, in a direction intersecting the main direction, each of the plurality of small regions each extending in a direction intersecting the main direction in the tomographic image. In this case, the tilt of the layer is appropriately reduced by parallel shift of each of the plurality of small regions.

The plurality of small regions forming the tomographic image may be selected as appropriate. For example, when a tomographic image is captured by an OCT device, pixel rows in a direction along the optical axis of OCT light in the tomographic image may be called as an A-scan image. In this case, a plurality of A-scan images constituting the tomographic image may constitute the small regions. Further, a plurality of pixel columns perpendicular to the A-scan image may constitute the small regions. Each small region may include a plurality of pixel columns.

Further, a specific method for aligning the plurality of small regions may also be selected as appropriate. For example, the control unit may align the plurality of small regions so that the positions of portions having maximum brightness in the plurality of small regions extending in the direction intersecting the main scanning direction are aligned with each other. The control unit may also detect a specific layer or a specific layer boundary (hereinafter, simply referred to as a “layer/boundary”) that appears in the tomographic image, and may aligning the positions of the plurality of small regions such that the detected layer/boundary extends linearly along the main direction. Alternatively, the control unit may detect the amount of positional deviation between adjacent small regions using a phase-only correlation method or template matching, and then arrange the plurality of small regions so that the detected amount of deviation is eliminated.

However, in addition to or in place of the above method, it is also possible to perform the tilt-reduction process using other methods. For example, the control unit may reduce the tilt of the layer with respect to the main direction by performing image processing such as rotation and shearing (skew) on the two-dimensional tomographic image. In this case, for example, an image processing method such as affine transformation may be used.

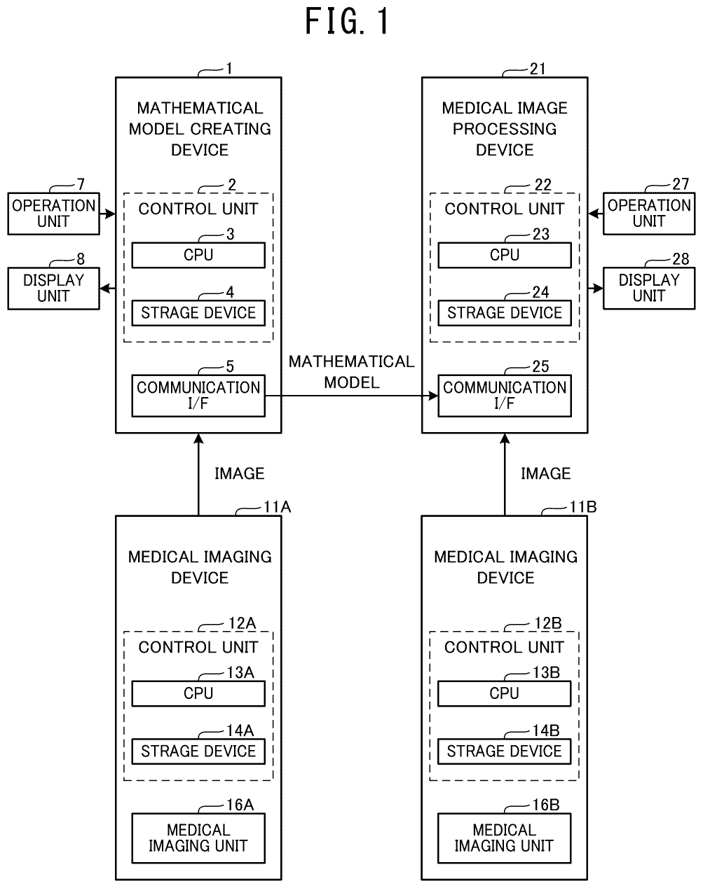

Hereinafter, one of a plurality of typical embodiments according to the present disclosure will be described with reference to the drawings. As shown in FIG. 1, in this embodiment, a mathematical model creating device 1, a medical image processing device 21, and medical imaging devices 11A and 11B are used. The mathematical model creating device 1 creates a mathematical model by training the mathematical model using a machine learning algorithm. The created mathematical model outputs medical data by processing input images. The medical image processing device 21 acquires medical data based on tomographic images using the mathematical model. The medical imaging devices 11A and 11B capture tomographic images of a living body's tissue (in this embodiment, a fundus tissue of a subject's eye).

As one example, a personal computer (hereinafter, referred to as a “PC”) is used as the mathematical model creating device 1 in this embodiment. Although details will be described later, the mathematical model creating device 1 creates the mathematical model by training the mathematical model using (i) images acquired from the medical imaging device 11A (hereinafter, referred to as “input data”) and (ii) medical data corresponding to the input data (hereinafter, referred to as “output data”). However, the device that may serve as the mathematical model creating device 1 is not necessarily limited to a PC. For example, the medical imaging device 11A may also serve as the mathematical model creating device 1. Further, control units of a plurality of devices (for example, a CPU of the PC and a CPU 13A of the medical imaging device 11A) may cooperatively create the mathematical model.

Furthermore, a PC is used as the medical image processing device 21 in this embodiment. However, the device that may serve as the medical image processing device 21 is not necessarily limited to a PC. For example, the medical imaging device 11B, a server, or the like may serve as the medical image processing device 21. When the medical imaging device (an OCT device in this embodiment) 11B serves as the medical image processing device 21, the medical imaging device 11B captures tomographic images of living tissue and also acquires medical data based on the captured tomographic images. Further, a mobile terminal device such as a tablet terminal device or a smartphone may serve as the medical image processing device 21. The control units of multiple devices (for example, the CPU of the PC and the CPU 13B of the medical imaging device 11B) may cooperatively perform various processes.

Next, the mathematical model creating device 1 will be described below. The mathematical model creating device 1 is placed, for example, at a manufacturer that provides the medical image processing device 21 or a medical image processing program to users. The mathematical model creating device 1 includes a control unit 2 that performs various control processes and a communication I/F 5. The control unit 2 includes a CPU 3, which is a controller, and a storage device 4 that is capable of storing programs, data, and the like. The storage device 4 stores a mathematical model creating program for executing a mathematical model creating process, as will be described later. Further, the communication I/F 5 connects the mathematical model creating device 1 to other devices (for example, the medical imaging device 11A and the medical image processing device 21, etc.).

The mathematical model creating device 1 is connected to an operation unit 7 and a display unit 8. The operation unit 7 is operated by a user in order for the user to input various instructions into the mathematical model creating device 1. As the operation unit 7, for example, at least one of a keyboard, a mouse, a touch panel, etc. may be used. Note that a microphone or the like for inputting various instructions may be used together with or in place of the operation unit 7. The display unit 8 displays various images. As the display unit 8, various devices capable of displaying images (for example, at least one of a monitor, a display, a projector, etc.) may be used. Note that an “image” in the present disclosure includes both still images and motion images.

The mathematical model creating device 1 acquires image data (hereinafter, may be sometimes simply referred to as an “image”) from the medical imaging device 11A. The mathematical model creating device 1 may acquire image data from the medical imaging device 11A through at least one of wired communication, wireless communication, a removable storage medium (for example, a USB memory), and the like.

Next, the medical image processing device 21 will be described. The medical image processing device 21 is placed, for example, in a facility (for example, a hospital or a medical examination facility) where diagnosis or examination of a subject is performed. The medical image processing device 21 includes a control unit 22 that performs various control processes, and a communication I/F 25. The control unit 22 includes a CPU 23, which is a controller, and a storage device 24 that is capable of storing programs, data, and the like. The storage device 24 stores a medical image processing program for executing a medical image process, as will be described later. The medical image processing program includes a program that realizes the mathematical model created by the mathematical model creating device 1. The communication I/F 25 connects the medical image processing device 21 to other devices (for example, the medical imaging device 11B, the mathematical model creating device 1, etc.).

The medical image processing device 21 is connected to an operation unit 27 and a display unit 28. As with the operation unit 7 and the display unit 8 described above, various devices can be used as the operation unit 27 and the display unit 28.

Each of the medical imaging devices 11 (11A, 11B) includes a control unit 12 (12A, 12B) that performs various control processes, and a medical imaging unit 16 (16A, 16B). The control unit 12 includes a CPU 13 (13A, 13B) that is a controller and a storage device 14 (14A, 14B) that can store programs, data, and the like.

The medical imaging unit 16 includes various components necessary for capturing tomographic images of a living tissue (in this embodiment, an ophthalmological image of the subject's eye). The medical imaging unit 16 in this embodiment includes an OCT light source, a branching optical element that branches OCT light emitted from the OCT light source into a measurement light and a reference light, a scanning section for scanning a target with the measurement light, an optical system for irradiating a subject's eye with the measurement light, and a light receiving element that receives the combined light of the light reflected by the tissue and the reference light.

The medical imaging devices 11 is capable of capturing a tomographic image (at least one of a two-dimensional tomographic image and a three-dimensional tomographic image) of a living tissue (in this embodiment, the fundus of a subject's eye). Specifically, the CPU 13 emits OCT light (the measurement light) on a scanning line to capture a two-dimensional tomographic image of a cross-section intersecting the scanning line. The two-dimensional tomographic image may be an averaged image generated by averaging a plurality of tomographic images on the same region. Furthermore, the CPU 13 may also capture a three-dimensional tomographic image of the tissue by performing two-dimensional scanning with the OCT light.

(Mathematical Model Creating Process)

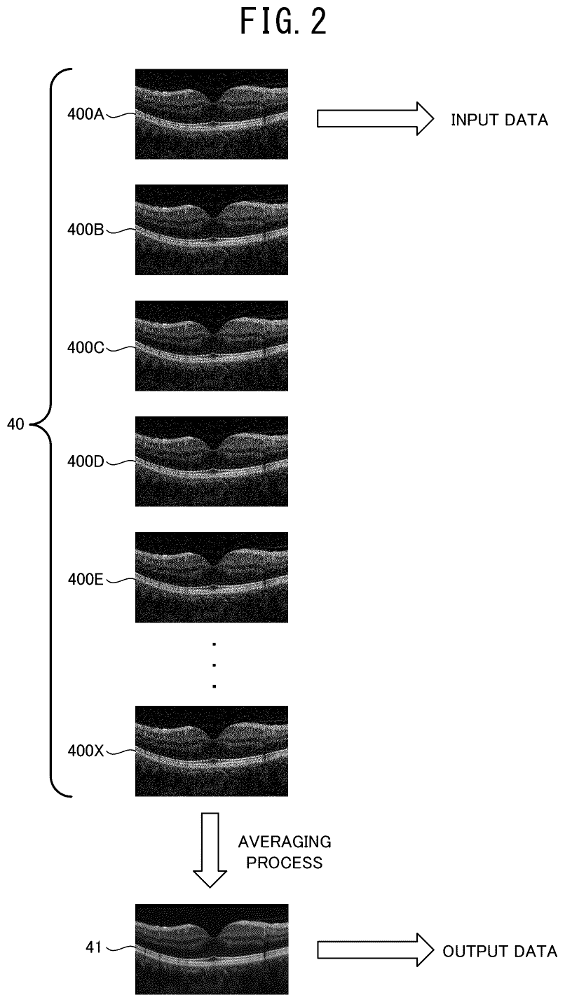

With reference to FIG. 2, the mathematical model creating process executed by the mathematical model creating device 1 will be described. The mathematical model creating process is executed by the CPU 3 according to the mathematical model creating program stored in the storage device 4.

In the mathematical model creating process, a mathematical model is trained using a plurality of pieces of training data, thereby developing a mathematical model to output medical data based on images. The training data includes data of the input side (i.e., input data) and data of the output side (i.e., output data). The mathematical model may output various medical data. The type of training data used to train the mathematical model may be selected depending on a type of medical data to be output by the mathematical model.

In this embodiment, by inputting a tomographic image (for example, a two-dimensional tomographic image) into a mathematical model as a base image, the mathematical model outputs, as medical data, a tomographic image (high-quality image) with improved quality based on the base image. In the present embodiment, a mathematical model is trained using a two-dimensional tomographic image of the tissue of the subject's eye as input data and a two-dimensional tomographic image of the same region with higher quality than the input data as output data. Note that the high-quality image refers to, for example, at least one of an image in which a noise in the input base image is reduced, an image in which the resolution of the original image is increased, an image in which the visibility of the original image is improved, and the like.

FIG. 2 shows one example of training data (i.e., input data and output data) where high-quality tomographic image data is output, as medical data, from a mathematical model. In the example shown in FIG. 2, the CPU 3 acquires a set 40 of a plurality of tomographic images 400A to 400X captured on the same region of the tissue. The CPU 3 uses some of the plurality of tomographic images 400A to 400X in the set 40 (the number of images is smaller than the number used for averaging output data which will be described later) as input data. Further, the CPU 3 obtains an average image 41 of the plurality of tomographic images 400A to 400X in the set 40 as output data. When a mathematical model is trained using the input data and the output data illustrated in FIG. 2, data of a high-quality image with reduced speckle noise influence is output as medical data by inputting the tomographic images as the base image into the trained mathematical model.

However, configuration of the mathematical model may be changed. For example, the mathematical model may perform an analysis process on at least one of a specific structure and a specific disease shown in the tomographic image, and output data indicative of analysis results as medical data. In this case, analysis results of at least one of layers of the fundus tissue of the subject's eye, boundaries of the layers of the fundus tissue, an optic disc present in the fundus, layers of an anterior segment tissue, boundaries of the layers of the anterior segment tissue, and a diseased area of the subject's eye may be output. Further, the mathematical model may perform an automatic diagnosis process on the tissue shown in the tomographic image, and output data indicative of automatic diagnosis results as medical data. Further, the mathematical model may output reliability information indicative of reliability of processing (for example, structure or disease analysis processing, etc.) performed on the input medical image as medical data. The type or form of the training data may be appropriately selected depending on the function of the mathematical model to be developed.

Next, the mathematical model creating process will be described. The CPU 3 acquires at least one of the tomographic images captured by the medical imaging device 11A as input data. Next, the CPU 3 acquires output data corresponding to the input data. An example of the correspondence between the input data and the output data is as described above. Next, the CPU 3 performs training of the mathematical model using the training data by a machine learning algorithm. As machine learning algorithms, for example, neural networks, random forests, boosting, support vector machines (SVM), etc. are generally known.

The neural network is a method that imitates the behavior of biological nerve cell networks. The neural network includes, for example, feedforward neural networks, RBF networks (radial basis functions), spiking neural networks, convolutional neural networks, recurrent neural networks (recurrent neural networks, feedback neural networks, etc.), and stochastic neural networks (Boltzmann machines, Bassian networks, etc.). Random forest is a method of generating a large number of decision trees by performing learning based on randomly sampled training data. When using a random forest, branches of multiple decision trees trained in advance as a classifier are followed, and the average (or the majority vote) of the results obtained from each decision tree is calculated.

Boosting is a method of generating a strong classifier by combining multiple weak classifiers. A strong classifier is created by sequentially training simple and weak classifiers. SVM is a method of creating a two-class pattern discriminator using linear input elements. The SVM learns the parameters of a linear input element based on the criterion (hyperplane separation theorem) of finding a margin-maximizing hyperplane that maximizes the distance to each data point from training data.

A mathematical model refers to, for example, a data structure for predicting the relationship between the input data and the output data. A mathematical model is created by being trained using training data. As described above, training data is a set of input data and output data. For example, correlation data (e.g., weights) between each input and output is updated by training.

In this embodiment, a multilayer neural network is used as a machine learning algorithm. A neural network includes an input layer for inputting data, an output layer for generating data to be predicted, and one or more hidden layers between the input layer and the output layer. A plurality of nodes (also called as “units”) are arranged in each layer. Specifically, in this embodiment, a convolutional neural network (CNN), which is one type of multilayer neural networks, is used. However, other machine learning algorithms may be used. For example, generative adversarial networks (GAN), which utilize two competing neural networks, may be used as the machine learning algorithm.

The above processes are repeated until the mathematical model is created. When the mathematical model is created, the mathematical model creating process ends. A program and data for realizing the created mathematical model are incorporated into the medical image processing device 21.

Note that the mathematical model used in this embodiment may be trained using training data that includes a tilt-reduction process (details will be described later) for reducing the tilt of the layer with respect to a main direction. In this case, the mathematical model is configured to output medical data with higher accuracy by inputting tomographic images in which the tilts of the layers with respect to the main direction is reduced.

(Medical Image Process)

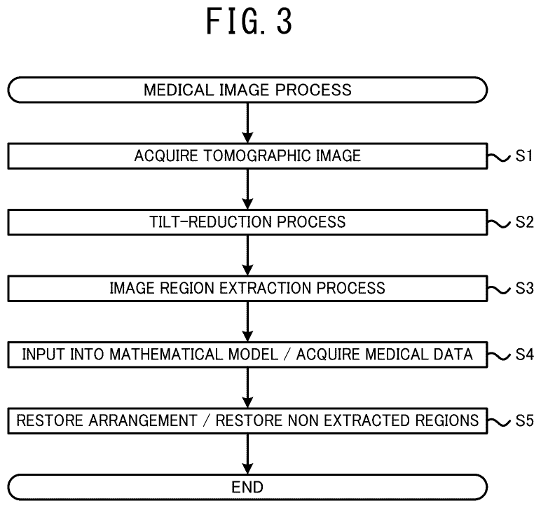

One example of the medical image process performed by the medical image processing device 21 will be described with reference to FIGS. 3 to 9. FIGS. 3 to 9 illustrate a situation in which two-dimensional tomographic image data of the fundus of the eye is processed using a mathematical model to obtain two-dimensional tomographic image data with higher quality than the original image before being processed. The medical image process illustrated in FIG. 3 is executed by the CPU 23 according to the medical image processing program stored in the storage device 24.

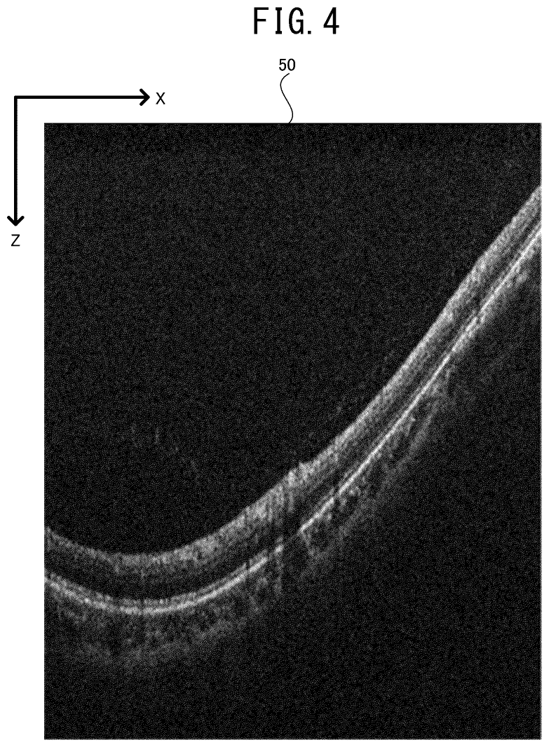

First, the CPU 23 acquires a tomographic image of the tissue of the subject's eye, which is photographed by the medical imaging device (the OCT device in this embodiment) 11B (S1). FIG. 4 shows one example of a tomographic image 50 captured by the medical imaging device 11B. The two-dimensional tomographic image 50 captured by the OCT device is formed of a plurality of A-scan images. The A-scan image is a plurality of pixel rows each extending in a direction along the optical axis of the OCT measurement light (that is, the Z direction which is the depth direction). In other words, a plurality of A-scan images each extending in the Z direction are arranged in the X direction (in this embodiment, the direction in which the spot of the OCT measurement light is emitted onto the tissue) perpendicular to the Z direction such that the two-dimensional tomographic image 50 is formed.

In the present disclosure, when a tomographic image of a specific tissue of a living body is captured, a direction in which the layers of the tissue that appear in the tomographic image generally extend is defined as a “main direction”. When a tomographic image of the fundus tissue of the subject's eye is captured by the medical imaging device 11B according to this embodiment, most of the layers of the fundus tissue that appear in the captured tomographic image usually extend in the X direction that is perpendicular to the Z direction along the optical axis of the OCT measurement light. For this reason, the main direction in this embodiment is the X direction.

The tomographic image 50 illustrated in FIG. 4 is an image of a fundus that has a greater degree of curvature than a normal fundus. Therefore, in the tomographic image 50 illustrated in FIG. 4, the angle (or the tilt) with respect to the main direction (the X direction) of the layer in a left side portion relative to the center is relatively small, but the angle (or the tilt) with respect to the main direction (the X direction) of the layer in a right portion relative to the center is significantly large.

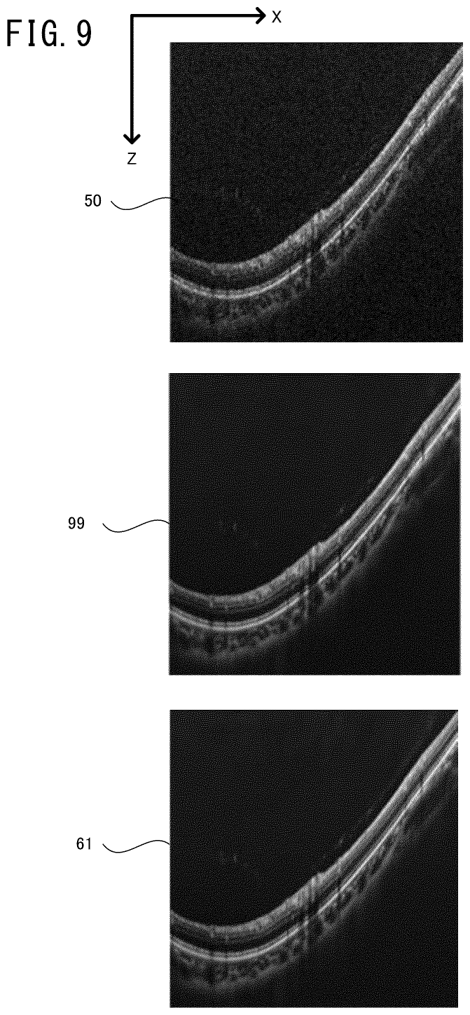

Although details will be described later with reference to FIG. 9, the inventors of the present disclosure have newly discovered that as the angle of the layer with respect to the main direction increases, the accuracy of the medical data output by the mathematical model decreases. During the medical image process in this embodiment, processing for acquiring medical data with higher accuracy is performed regardless of the angle of the layer with respect to the main direction. The details of the process will be described below.

Returning back to FIG. 3, the CPU 23 executes a tilt-reduction process on the tomographic image 50 acquired at S1 (S2). The tilt-reduction process is a process to reduce the tilts of layers in the tomographic image with respect to the main direction (X direction).

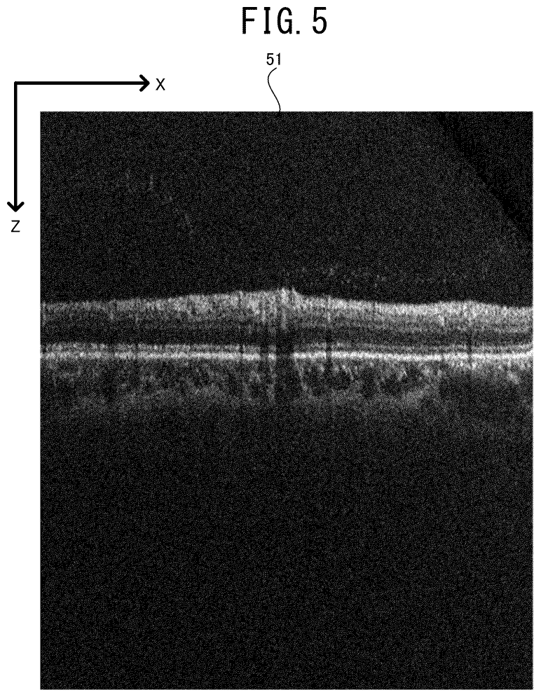

FIG. 5 shows the result after the tilt-reduction process was performed on the tomographic image 50 shown in FIG. 4. As shown in FIG. 5, in a tilt-reduced image 51 on which the tilt-reduction process has been performed, the curvature (or the curved shape) of the layer that appeared in the tomographic image 50 shown in FIG. 4 is reduced, and the layer is flattened in the X direction.

Specifically, in the tilt-reduction process (S2) in this embodiment, the tomographic image 50 is divided into a plurality of small regions (in this embodiment, a plurality of A-scan images) each of which extends in the Z direction perpendicular to the main direction (the X direction). Then, the CPU 23 aligns the positions of images in the Z direction appearing in the plurality of small regions of the tomographic image 50 with each other by moving (or shifting) the images in the small regions in the Z direction. As a result, the tilt of the layer with respect to the main direction is appropriately reduced. Note that the direction of movement (a movement direction) and the amount of movement of each A-scan image are stored in the storage device 24 to be used during an arrangement restoration process (S5), as will be described later.

At S2 in the present embodiment, the CPU 23 detects a specific layer or a boundary between layers in the tomographic image 50 and aligning the positions of the images in the small regions such that the detected specific layer or the detected boundary extends substantially linearly (in other words, have a linear shape) along the main direction (i.e., the X direction). However, the method for aligning the images in the plurality of small regions may be changed as appropriate. For example, the CPU 23 may move each of the plurality of small regions in the Z direction such that the positions of portions each having a maximum brightness in the plurality of small regions are aligned with each other in the Z direction. Alternatively, the CPU 23 may detect the amount of positional deviation between adjacent small regions using a phase-only correlation method or template matching, and then arrange the plurality of small regions so that the detected amount of deviation is eliminated.

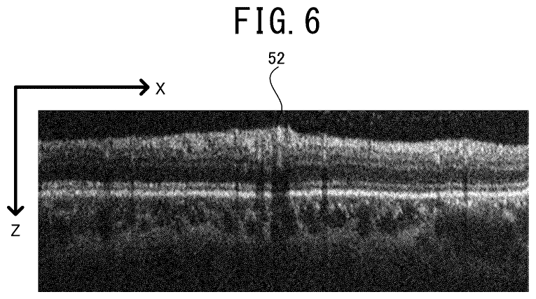

Next, the CPU 23 executes an image region extraction process for extracting an image region in which the tissue appears from the tomographic image (S3). In this embodiment, after the tilt-reduction process (S2) was performed on the tomographic image, the image region extraction process (S3) is performed. That is, the extracted image 52 shown in FIG. 6 is an image that was extracted from the tilt-reduced image 51 shown in FIG. 5. However, the CPU 23 may perform the tilt-reduction process after performing the image area extraction process on the tomographic image 50 acquired at S1. As shown in FIG. 6, the data amount of the extracted image 52 is smaller than the data amount of the tomographic image before the image region extraction process is performed. As a result, the amount of calculation for processing using the mathematical model (details will be described later) is appropriately reduced.

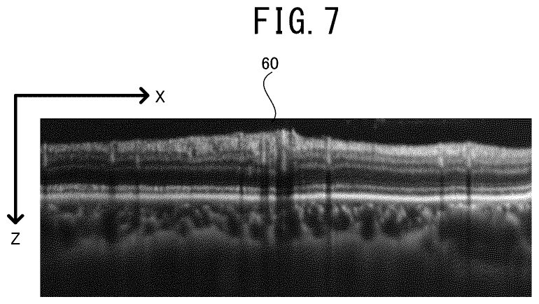

Next, the CPU 23 inputs the tomographic image that were subject to the tilt-reduction process (specifically, the extracted image 52, which is a tomographic image that has undergone both the tilt-reduction process and the image area extraction process) into the mathematical model to acquire medical data (S4). As described above, the mathematical model exemplified in this embodiment performs processing on the input tomographic image (i.e., the base image) to generate high-quality image data, as the medical data, with improved quality for medical purposes. The CPU 23 acquires high-quality image data output by the mathematical model. FIG. 7 shows a high-quality image 60 output by the mathematical model based on the extracted image 52 shown in FIG. 6. The quality of the high-quality image 60 (see FIG. 7) is improved as compared to the quality of the extracted image 52 (see FIG. 6) prior to being input into the mathematical model.

As described above, the tomographic image input into the mathematical model in S4 has been subjected to the tilt-reduction process (S2). As a result, compared to a situation where the tomographic image 50 itself is input into a mathematical model without performing the tilt-reduction process, it is possible to avoid decreasing in accuracy of medical data (the high-quality image data in this embodiment) due to the tilt of the layer with respect to the main direction (the X direction).

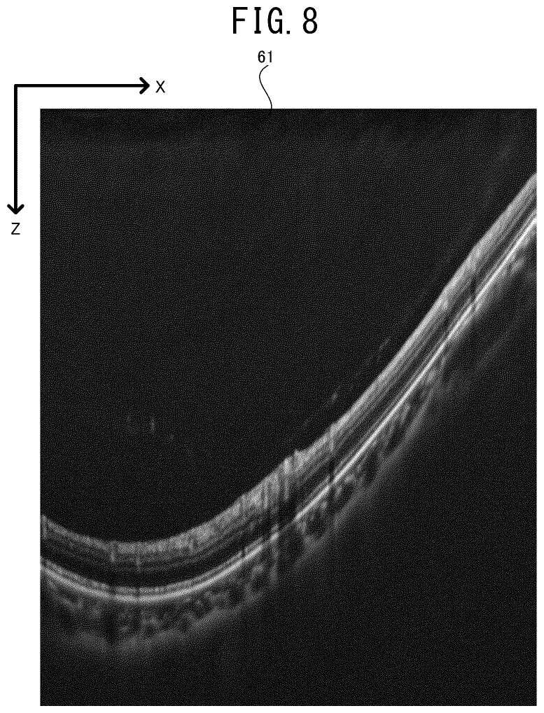

Next, the CPU 23 executes the arrangement restoration process and a non-extracted region restoration process (S5). In the arrangement restoration process, the CPU 23 restores the arrangement of the high-quality image 60 to an original arrangement before the tilt-reduction process was performed by performing an opposite process (i.e., a reverse process) to the tilt-reduction process (S2) on the medical data (i.e., the data of the high-quality image 60) acquired at S4. In this embodiment, the CPU 23 restores the arrangement of each of the small regions by moving each of the small regions by the amount of movement at S2 in a direction opposite to the direction of movement in which each of the small regions (i.e., the A-scan images) were moved during the tilt-reduction process (S2). In addition, at the non-extracted area restoration process, the CPU 23 restores regions that were not extracted at the image area extraction process (S3) on the high-quality image 60 acquired at S4, thereby increasing the size of the high-quality image to an original size before the image region was extracted. FIG. 8 shows a restored image 61 acquired by performing the arrangement restoration process and the non-extracted area restoration process on the high-quality image 60 shown in FIG. 7.

Next, advantageous effects of the present disclosure will be described with reference to FIG. 9. The uppermost image in FIG. 9 is a tomographic image 50 immediately after being captured by the medical imaging device 11B (that is, the tomographic image 50 shown in FIG. 4 before being subject to the quality improvement process using the mathematical model). The middle image in FIG. 9 is a high-quality image (i.e., a comparative image 99) acquired by inputting the tomographic image 50 as it is into the mathematical model without performing the tilt-reduction process (S2) on the tomographic image 50. The lowermost image in FIG. 9 is a high-quality image (that is, the restored image 61 shown in FIG. 8) acquired by performing the processes S2 to S4 on the tomographic image 50.

As shown in FIG. 9, both the comparative image 99 and the restored image 61 have improved image quality to the same extent in the left sides of the images 99, 61 with respect to the center as compared to the uppermost tomographic image 50. This is because the tilt of the layer with respect to the main direction (X direction) is relatively reduced on the left side portion. On the other hand, the right side portion of the restored image 61 has further improved quality as compared to the comparative image 99. From the above results, the medical data can be acquired with higher accuracy by inputting an image that has been subject to the tilt-reduction process into a mathematical model, regardless of the tilt of the layer with respect to the main direction.

The techniques disclosed in the above embodiments are merely examples. Therefore, it is also possible to modify the techniques exemplified in the above embodiments. First, only a part of the processes exemplified in the above-described embodiment may be executed. For example, in the medical image process shown in FIG. 3, at least one of the image region extraction process (S3) and the restoration process (S5) may be omitted. Further, the mathematical model is not necessarily limited to a mathematical model that is configured to output high-quality image data as the medical data.

The CPU 23 may also extract a two-dimensional tomographic image from a three-dimensional tomographic image and acquire, using a mathematical model, medical data (for example, high-quality image data, etc.) based on the extracted two-dimensional tomographic image. In this case, the CPU 23 may perform the tilt-reduction process on the extracted two-dimensional tomographic image and input it into the mathematical model. Further, the CPU 23 may execute the tilt-reduction process to reduce the tilt of the layer with respect to the main direction on the three-dimensional tomographic image, and then extract the two-dimensional tomographic image from the three-dimensional tomographic image and input the extracted image into the mathematical model. In this case, it is not necessary to perform the tilt-reduction process each time a two-dimensional tomographic image is extracted. For example, the main direction for processing a three-dimensional tomographic image of the fundus captured by an OCT device may be the X-Y direction perpendicular to the tissue depth direction (i.e., the Z direction). A method for extracting a two-dimensional tomographic image from a three-dimensional tomographic image may be arbitrarily selected. For example, when a three-dimensional tomographic image is viewed in a direction along the optical axis of the imaging light (e.g., OCT light), the two-dimensional tomographic image may be extracted such that the position at which the two-dimensional tomographic image is extracted has a circle or cross shape.

The process of acquiring a tomographic image at S1 of FIG. 3 is one example of an “image acquisition step.” The tilt-reduction process at S2 is one example of a “tilt-reduction step.” The process of acquiring medical data at S4 is one example of a “medical data acquisition step.” The restoration process executed at S5 is one example of a “restoration step.” The image region extraction process executed at S3 is one example of an “image region extraction step.”

1. A medical image processing device that processes tomographic image data of a living tissue, the device comprising:
a control unit that includes at least one processor and at least one memory storing computer program code, the computer program code, when executed by the at least one processor, causing the at least one processor to:
acquire a tomographic image in which a layer of the living tissue appears;
perform a tilt-reduction process on the acquired tomographic image to reduce a tilt of the layer of the living tissue with respect to a main direction; and
acquire medical data by inputting, into a mathematical model, a tilt-reduced image that is the tomographic image on which the tilt-reduction process was performed, wherein
the mathematical model is trained by a machine learning algorithm to output medical data by processing an input image.

2. The medical image processing device according to claim 1, wherein
when an image is input, the mathematical model is configured to output, as the medical data, data of a high-quality image with improved quality with respect to the input image.

3. The medical image processing device according to claim 1, wherein
the at least one processor is further caused to restore an arrangement of the medical data to an original arrangement of the medical data prior to the tilt-reduction process being performed by performing, on the acquired medical data output from the mathematical model, an opposite process to the tilt-reduction process.

4. The medical image processing device according to claim 1, wherein
the at least one processor is further caused to:
extract, from the tomographic image, an image region in which the tissue appears; and
input, into the mathematical model, the image region of the tomographic image on which the tilt-reduction process was performed.

5. The medical image processing device according to claim 1, wherein
the tomographic image is formed of a plurality of small regions each extending in a direction intersecting the main direction, and
the at least one processor is further caused to align positions of the plurality of small regions with each other by moving the plurality of small regions in the direction intersecting the main direction.

6. A non-transitory, computer readable storage medium storing a medical image processing program executed by a medical image processing device that processes tomographic image data of a living tissue, the program, when executed by at least one processor of the medical image processing device, causing the at least one processor to:
acquire a tomographic image in which a layer of the living tissue appears;
perform a tilt-reduction process on the acquired tomographic image to reduce a tilt of the layer of the living tissue with respect to a main direction; and
acquire medical data by inputting, into a mathematical model, a tilt-reduced image that is the tomographic image on which the tilt-reduction process was performed, wherein
the mathematical model is trained by a machine learning algorithm to output medical data by processing an input image.

7. A medical image processing method implemented by a medical image processing device that processes tomographic image data of a living tissue, the method comprising:
acquiring a tomographic image in which a layer of the living tissue appears;
performing a tilt-reduction process on the acquired tomographic image to reduce a tilt of the layer of the living tissue with respect to a main direction; and
acquiring medical data by inputting, into a mathematical model, a tilt-reduced image that is the tomographic image on which the tilt-reduction process was performed, wherein
the mathematical model is trained by a machine learning algorithm to output medical data by processing an input image.
